(19) United States
(12) Patent Application Publication
(10) Pub. No.: US 2021/0015652 A1
(43) Pub. Date: Jan. 21, 2021

(54) Orthotic Insert

(71) Applicant: Ryan Montgomery, Lexington, KY (US)

(72) Inventor: Ryan Montgomery, Lexington, KY (US)

(21) Appl. No.: 16/512,353

(22) Filed: Jul. 15, 2019

(51) Int. Cl. A61F 5/14

(52) U.S. Cl. CPC A61F 5/14; A43B 7/1415

(57) ABSTRACT

A half-orthotic insert beginning at the heel and extending to the metatarsal region of the foot having raised elements that align with the fascia ligament for the purpose of providing relief of plantar fasciitis.

### RELATED APPLICATION

This application claims priority from U.S. Provisional 62/697,949 filed Jul. 13, 2019.

### TECHNICAL FIELD

This present invention relates generally to orthotic inserts. More particularly, the present invention relates to a corrugated methodology to support the fascia. The relevant CPC classifications are:
A61F 5/14 . . . Special medical insertions for shoes for flat-feet, club-feet, or the like
A43B 7/14 . . . Footwear with foot-supporting parts

### BACKGROUND

### Description of Relevant Art

The human walking system is a highly complex interaction of more than 28 bones, 30 joints where sinew in the form of both muscle and ligament work with bone, blood flow, and nerves to allow a human to take over 100 million steps in a lifetime. It is not surprising that inventions have been developed over time to make walking and standing on our feet more comfortable. U.S. Pat. No. 155,187 was one of the first “elevate your feet” innovations. U.S. Pat. No. 613,904 was one of the first devices to improve gait based on podiatric science. Over time, heel support (U.S. Pat. No. 3,985,853), arch support and other methods have been developed to reduce stress on the foot walking system (see U.S. Pat. Nos. 6,745,501 and 5,404,659). Some of these orthotic devices can be customized; some are retail-ready focusing on not having to design multiple sizes (U.S. Pat. No. 7,958,653) while still cushioning the blow of each and every step. Some inventions have focused on special orthotic insert materials (such as a movable fluid or gel: U.S. Pat. No. 7,784,197).

In this present invention, we present a orthotic support system that relies on corrugated technology (see U.S. Pat. No. 4,286,006). Common to cardboard, this technology creates natural rigidity and resistance to compressive forces. In short, an orthotic system using corrugated technology will reduce strain on the fascia ligament. A human step consists of three phases: the heel strike, the stance phase, and the toe off. The fascia ligament is the connective tissue between the heel bones and the metatarsals. Unlike muscles, ligaments do not stretch, so if this ligament gets stressed it can cause all sorts of maladies: heel pain, bone spurs, micro tears, and even lower-leg blood clots or possibly deep-vein thrombosis.

The human body works best when all systems function properly: bones, joints connected with soft tissues such as ligaments or muscles, blood-flow to properly supply nutrients to the soft tissues, and the nervous system that provides feedback on the systems proper functioning. The present invention indirectly supports all of these functions related to the important fascia ligament. Similarly to U.S. Pat. No. 6,315,786 the present invention uses a bar support system that runs perpendicular(ish) to the direction of the stress placed on the fascia. This present invention uses a plurality of such bar supports in a corrugated fashion to support the fascia (from distal to the calcaneus ending near the metatarsal region), randomize the force of the blow evenly across the entire fascia, massage the fascia region (including the nearby arteries and nerves) creating a unique “rumble-strip effect” for the foot at each step. Clients that have used this technology have commented on improved balance, less pain, faster recovery from fatigue, more energy, fewer circulatory problems in the lower leg/foot region, and so on.

### ADVANTAGES

Our speed-bump “rumble-strip” methodology using corrugated technologies has several advantages: it dissipates the downward/forward forces associated with each step by deflecting into randomized directions; it has spacing to allow these forces to reduce the overall shock load on the fascia ligament; this corrugation pattern of structure and space creates micro vibrations that directly message the fascia while also influencing the adjacent arterial and nerval systems.

### SUMMARY

In accordance with one embodiment, the preferred embodiment, comprising of a half-orthotic insert beginning at the heel and extending to the metatarsal region of the foot. A very soft silica plastic is one material for use, but the material is not limited to plastic could include: fibers, nylons, gels. In addition the half-orthotic insert could also be altered to be other embodiments such as socks, full inserts, custom inserts. Custom orthotics could have mineral materials supplementing the primary body structure: copper, silver, titanium, and so on. The primary design would run perpendicular to the direction of the fascia heel-strike/stance-phase/toe-off, but variations could run radial to the inner arch to further support the fascia in regards to pronation and supination.

### DRAWINGS—FIGURES

In the drawings, closely-related figures have the same number but different alphabetic suffixes.

FIG. 1 shows the plantar fascia of the foot with an emphasis of the direction of the force displaced for each step.

FIG. 2a shows an example embodiment with a heel base, and four bar supports (blue) with space between each. The support bars run perpendicular(ish) to the force from FIG. 1.

FIG. 2b shows an example bar-support pattern with five supports.

FIG. 2c shows an example bar-support patter with five supports radially from the instep (arch).

FIG. 3 shows potential profile views of the rumble-strip methods with varying patterns.

### DRAWINGS—DETAILED DESCRIPTION

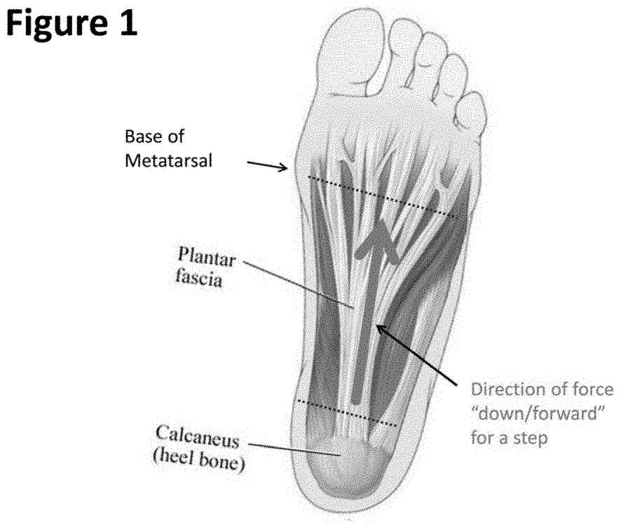

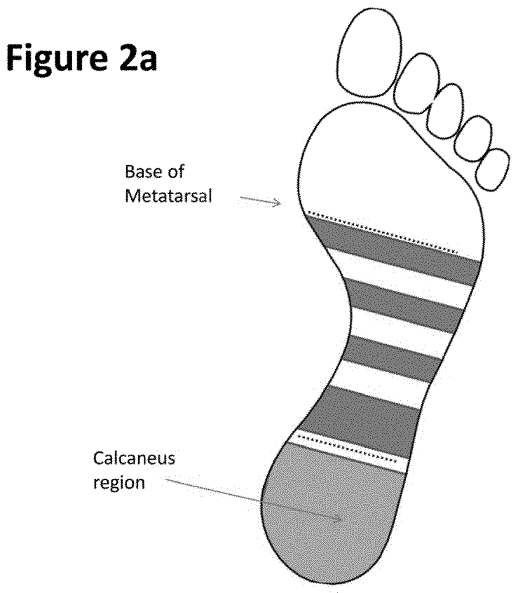

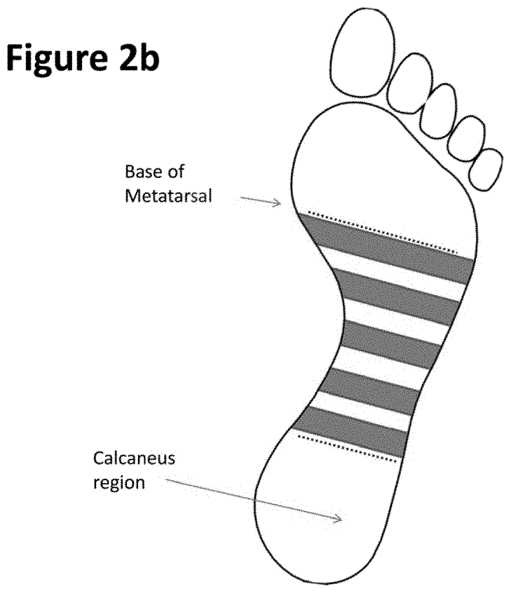

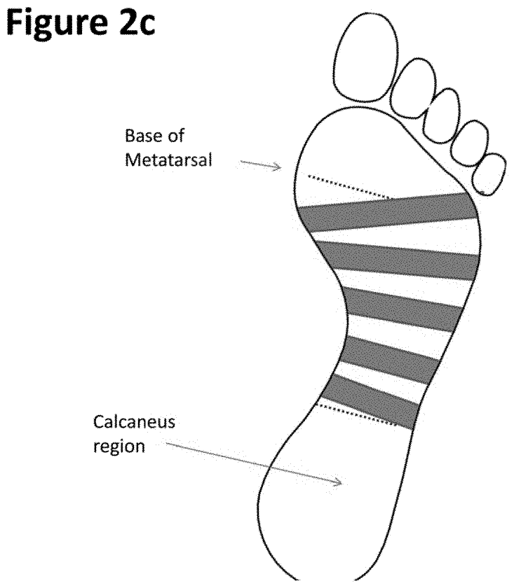

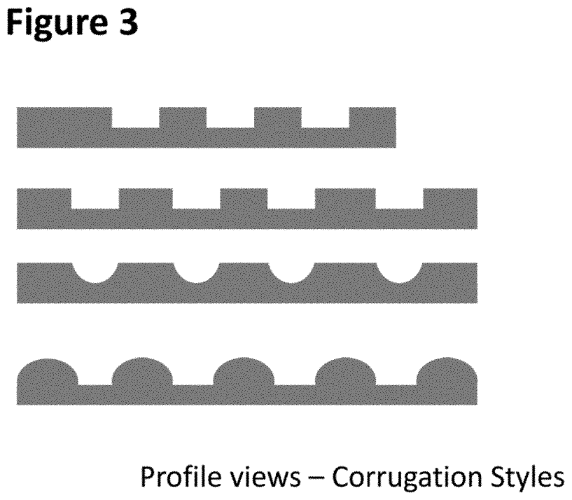

In FIG. 1, we outline the nature of the force on the plantar fascia (down-forward) for each step taken. The direction of the force is aligned with the natural formation of the fascia ligament. The fascia connect the heel bones to the metatarsals. Supporting the fascia are both arterial and nerval systems. In FIG. 2a, we describe some basic elements of a preferred half-orthotic embodiment of this corrugated orthotic. This embodiment has a heel cup (in green, the calcaneus region) that allows the orthotic to be firmly located within a shoe; and extends to the base of the metatarsals. The speed-bump rumble strips are colored in blue and intentionally are set perpendicular(ish) to the force of the down-forward step motion. The bump pad and related space are on the top-side of the orthotic coming into direct contact with the foot (with our without hosiery). In this pattern, the first bump pad is approximately one inch in height, made of PPT/plastazote foam, about ⅛^{th} of an inch thick. The remaining bump pads are approximately ½ inch in height, made of a similar material, and each bump pad is separated by about ½ inch of space. The footbed connects the heel plate to all of the bump pads to create the overall orthotic device. In FIG. 2b, we disclose an alternative pattern for the bump pads, each ½ inch in height with a ½ inch space. In FIG. 2c, we disclose a variation of FIG. 2b, where the perpendicular(ish) direction of the bump pads are radially offset based on the inner arch to support pronation and possibly alternative variations could also support supination. In FIG. 3, we show profile views of the variations of the rumble-strip pad patterns where the bump pad, the space, and the attached footbed are shown together.

### Operation

This orthotic device is placed firmly in a pair of shoes (one for the left foot, one for the right foot). The user then performs normal daily activities with the orthotic in place, from simple ambulatory activity to more complex sport regimes.

Research literature has conflicting opinion about the ability to support the facia with orthogonal bracing using a technique called low-dye taping. This present invention provides an approach anchored to this orthogonal-benefit hypothesis.

### CONCLUSION, RAMIFICATION, AND SCOPE

Thus the reader will see that at least one embodiment of the facia support corrugated orthotic will provide relief to the foot pain associated with stress on the plantar facia and even those suffering from micro tears, plantar fasciitis, bone spurs, and so on.

While the above description contains much specificity, these should not be construed as limitations on the scope, but rather as examples of preferred embodiments. Accordingly, the scope should not be determined by the descriptions of the embodiments illustrated, but by the forthcoming legal claims.

What is claimed is:

1. An orthotic insert comprising raised areas that correspond with the fascia ligament of the foot for the purpose of relieving plantar fasciitis.
